(19) United States
(12) Patent Application Publication
(10) Pub. No.: US 2026/0301881 A1
(43) Pub. Date: Oct. 1, 2026

(54) SYSTEMS AND METHODS FOR DOSAGE PREDICTION

(71) Applicant: The University of Melbourne, Victoria (AU)

(72) Inventors: Justin Ming Chi YEUNG, Melbourne (AU); Paul Nigel BAIRD, Melbourne (AU); Ke CAO, Melbourne (AU)

(73) Assignee: The University of Melbourne, Victoria (AU)

(21) Appl. No.: 19/479,221

(22) PCT Filed: Apr. 26, 2024
(86) PCT No.: PCT/AU2024/050408

§ 371 Date: Oct. 27, 2025

(30) Foreign Application Priority Data
Apr. 28, 2023 (AU) 2023901263

(51) Int. Cl. G16C 20/30; G06T 7/00; G06T 7/10; G16C 20/70; G16H 20/10; G16H 30/40

(52) U.S. Cl. CPC G16C 20/30; G06T 7/0012; G06T 7/10; G16C 20/70; G16H 20/10; G16H 30/40; G06T 2207/10081; G06T 2207/30008

(57) ABSTRACT

Described embodiments relate to a method for predicting a toxicity of a drug to be administered to a patient. The method comprises accessing at least one computed tomography (CT) torso slice associated with the patient; using a trained artificial intelligence (AI) segmentation model to perform a labelling process of the at least one CT scan; determining at least one body composition parameter based on the at least one labelled CT slice; receiving at least one demographic parameter associated with the patient; receiving at least one dosage parameter associated with the drug to be administered to the patient; and using a trained AI prediction model, generating an output based on the at least one body composition parameter, the at least one demographic parameter and the at least one dosage parameter, the output corresponding to a predicted toxicity of the drug to the patient.

### TECHNICAL FIELD

Described embodiments generally relate to systems and methods for dosage prediction. In particular, described embodiments relate to systems and methods for performing drug dosage prediction to minimise adverse reactions to the drug.

### BACKGROUND

The unpredictability of the development of toxicities in a patient following a drug treatment, such as chemotherapy, is significant. For example, in a cohort of colonic cancer patients treated with Oxaliplatin, over 50% of patients developed severe or life threatening toxicities to the drug.

The dosage of chemotherapy to administer for treating cancer is often calculated crudely, based on parameters such as the patient's body surface area. This leads to a very high incidence of toxicities with chemotherapy. These toxicities cannot be accurately predicted and therefore lead to patients developing severe complications including sepsis and neuropathies, requiring patients to be admitted to hospital for treatment for those who develop severe problems. This overdosing occurs because patients with the same body surface area (BSA) may have very different body composition and therefore absorb the chemotherapy doses very differently.

There is a need for better methods of selecting a dose of a particular drug to be given to a particular patient to minimise the chances, or reduce the severity of, an adverse reaction to the drug in that patient.

It is desired to address or ameliorate one or more shortcomings or disadvantages associated with prior systems and methods for dosage prediction, or to at least provide a useful alternative thereto. Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

### SUMMARY

There is provided a method for predicting the toxicity of a drug to be administered to a patient. The method comprises:
receiving at least one computed tomography (CT) torso slice associated with the patient;
using a trained artificial intelligence (AI) segmentation model to perform a labelling process of the at least one CT scan;
determining at least one body composition parameter based on the at least one labelled CT slice;
receiving at least one demographic parameter associated with the patient;
receiving at least one dosage parameter associated with a drug to be administered to the patient; and
using a trained AI prediction model, generating an output based on the at least one body composition parameter, the at least one demographic parameter and the at least one dosage parameter, the output corresponding to a predicted toxicity of the drug to the patient.

In some embodiments, the method further comprises training the AI prediction model.

In some embodiments, the labelled CT slices include one or more or all of labelled areas of muscle, visceral adipose tissue (VAT), subcutaneous adipose tissue (SAT) and intermuscular/intramuscular adipose tissue (IMAT).

In some embodiments, the body composition parameter is at least one or more or all of an amount of muscle, an amount of VAT, an amount of SAT, an amount of IMAT, a radio-density of muscle, a radio-density of VAT, a radio-density of SAT or a radio density of IMAT.

There is provided a method for training an artificial intelligence model to assess a drug dosage for minimising a toxic reaction to the drug. The method comprises:
receiving a sample of labelled CT torso slices associated with a sample of patients;
determining at least one body composition parameter based on each labelled CT slice;
identifying a set of prediction parameters for each patient, the prediction parameters including the at least one body composition parameter, at least one patient demographic parameter and at least one dosage parameter;
for each combination of the prediction parameters, performing a model fitting process to cause the model to generate a predicted toxicity rating for each patient;
for each combination of the prediction parameters, comparing the predicted toxicity rating with a measured toxicity rating to determine the accuracy of the combination of prediction parameters; and
selecting the optimal combination of prediction parameters, wherein the optimal combination of prediction parameters are the combination of prediction parameters that result in the highest accuracy.

In some embodiments, the labelled CT slices include one or more or all of labelled areas of muscle, VAT, SAT and IMAT.

In some embodiments, the body composition parameter is at least one or more or all of an amount of muscle, an amount of VAT, an amount of SAT, an amount of IMAT, a radio-density of muscle, a radio-density of VAT, a radio-density of SAT, a radio-density of IMAT.

In some embodiments, the method further comprises using the trained AI model to perform labelling of a sample of CT slices to generate the sample of labelled CT slices.

In some embodiments, the CT slice is from the abdomen.

In some embodiments, between two and one thousand CT slices are received.

There is provided a method of minimising a toxic reaction to a drug in a patient. The method comprises:
i) determining the amount of muscle, visceral adipose tissue (VAT), subcutaneous adipose tissue (SAT) and intermuscular/intramuscular adipose tissue (IMAT) in at least a part of the torso of the patient,
ii) determining the risk of a toxic reaction in the patient to the drug at least based on i), and
iii) reducing a standard recommended dose of the drug if it is determined that the patient is at risk of a toxic reaction.

In some embodiments, the method further comprises administering the drug.

In some embodiments, step i) is performed using any of the above methods.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### BRIEF DESCRIPTION OF DRAWINGS

FIG. 1 shows an example flowchart of a method for dosage prediction according to some embodiments;

FIG. 2 illustrates an example architecture that may be used to perform the method of FIG. 1, in some embodiments;

FIG. 3A illustrates a first example labelled CT image taken of a first patient;

FIG. 3B illustrates a second example labelled CT image taken of a second patient;

FIG. 4 illustrates an example pipeline of a method for assessing toxicity for a specific patient according to some embodiments;

FIG. 5 shows a graph illustrating the accuracy of dosage prediction for different combinations of features; and

FIG. 6 shows an example system that may be used to perform the steps of FIGS. 1 and 4, according to some embodiments.

### DESCRIPTION OF EMBODIMENTS

Described embodiments generally relate to systems and methods for dosage prediction. In particular, described embodiments relate to systems and methods for performing drug dosage prediction to minimise adverse reactions to the drug.

Some embodiments relate to a tool for predicting the dose limiting toxicity (DLT) of a drug. Examples include drugs to treat cancer, which may include breast cancer, lung cancer, liver cancer, colon cancer, bowel cancer, prostate cancer, colorectal cancer, and/or pancreatic cancer. Further examples include drugs to treat diabetes, high blood pressure, pain, an autoimmune disease and/or neurodegenerative disease. Described embodiments may assist in balancing the achievement of a positive response to the drug, such as a reduction in the condition for which the drug is being administered, with the reduction in at least one side effect such as toxicity, neuropathy or another adverse reaction. The drug can be any type of substance administered to a patient to treat or prevent a condition. Examples of the types of drugs may include, but are not limited to, small molecules, antibodies, vaccines and viruses.

The absorption of drugs by patients is based on a number of factors, including the body composition of the patient. This may be estimated using measures such as the patient's height, weight, BMI and/or body surface area (BSA). However, none of these measures will accurately predict how a patient's body will absorb a particular drug.

More accurate measures of body composition can be derived from computed tomography (CT) scans. Trained practitioners can analyse a CT scan and determine which areas of the scan relate to specific tissue types, such as muscle, visceral adipose tissue (VAT) and subcutaneous adipose tissue (SAT), for example. However, this manual process is time consuming and tedious, and so is generally only used for research purposes. Furthermore, the measures of specific tissue in one CT scan image may not allow for an accurate derivation of the full body composition of the patient.

By automating the process of analysing CT scans, described embodiments allow for multiple CT slices from a single patient to be efficiently analysed via a segmentation process, allowing for a more accurate body composition to be calculated and a more accurate dosage to be predicted. The disclosed method further increases the accuracy of prediction by taking into account demographic and biometric parameters.

While some examples herein relate specifically to colorectal cancer chemotherapy, it is noted that the disclosed methods are equally applicable to other cancer types and other diseases, including breast cancer, lung cancer, liver cancer, colon cancer, bowel cancer, prostate cancer, pancreatic cancer, diabetes, high blood pressure, pain, autoimmune disease and/or neurodegenerative disease. More particularly, the segmentation method of the CT scans to calculate the body composition remains the same for all drugs. The final model that maps those body composition parameters to a toxicity or dosage can be trained individually for each drug as disclosed herein.

The term “patient” used herein refers to a mammal including humans, livestock such horses, cows, sheep and goats, and companion animals such as dogs and cats. In some embodiments, the patient is a human.

A computed tomography scan (usually abbreviated to CT scan; formerly called computed axial tomography scan or CAT scan) is a medical imaging technique used to obtain detailed internal images of the body. Some CT scanners use a rotating X-ray tube and a row of detectors placed in a gantry to measure X-ray attenuations by different tissues inside the body. The multiple X-ray measurements taken from different angles are then processed on a computer using tomographic reconstruction algorithms to produce tomographic (cross-sectional) images (virtual “slices”) of a body. Accordingly, when reference is made herein to a “CT scan”, this refers to the entire dataset generated by the computer for one patient and can contain one or more “CT slices”. A CT slice is an individual image representing a cross-section of the patient's body.

This disclosure provides a computer implemented method of assessing a drug dose using multiple different artificial intelligence (AI) models. These models may equally be described as machine learning (ML) models because there is a training process that tunes the internal parameters of the model so that the model output best corresponds to the labels of the training data.

A first model is referred to as the segmentation model. The segmentation model is trained to identify areas of tissue shown on a CT slice, which may include muscle tissue, visceral adipose tissue (VAT), subcutaneous adipose tissue (SAT), intermuscular/intramuscular adipose tissue (IMAT), bone and organ tissue. The segmentation model may be trained to perform this process by providing the model with training data comprising a series of manually labelled CT slices. Once trained, the segmentation model is provided new CT slices and performs an automatic labelling process. CT slices at a range of positions from a single patient can be provided to the model and labelled. In some embodiments, at least some of the slices are of the torso of the patient. In some embodiments, at least some of the slices are from a head, arm or leg of the patient. In some embodiments, slices are taken from between the neck and coccyx of the patient. In some embodiments, slices are taken between the T1 and T12 vertebrae. In some embodiments, slices are taken between the L1 and L5 vertebrae. In some embodiments, at least some of the slices are taken from the abdomen of the patient. In some embodiments, between two and 1,000 slices are analysed. In some embodiments, between 2 and 500 slices are analysed. In some embodiments, between 2 and 100 slices are analysed. In some embodiments, between 2 and 50 slices are analysed. In some embodiments, between 2 and 25 slices are analysed. In some embodiments, between 2 and 10 slices are analysed.

A number of body composition parameters may then be calculated based on the labelled CT scan data. These may include one or more of the surface area of muscle, VAT, SAT, IMAT, bone and/or organ tissue; the radio-density of muscle, VAT, SAT, IMAT, bone and/or organ tissue; the volume of muscle, VAT, SAT, IMAT, bone and/or organ tissue; a skeletal muscle index; and/or lean body mass. A skeletal muscle index (SMI, cm²/m²) may be calculated by mid-L3 muscle surface area (in cm²)/(Height (in m)\hat{ }2). An SMI value of <38.5 cm²/m² in women and <52.4 cm²/m² in men may be classified as being sarcopenic. Lean body mass (LBM) may be calculated as [((mid-L3 muscle surface area (in cm2)×0.3)+6.06].

A second model is referred to as the prediction model. The prediction model is trained to provide a toxicity prediction based on a number of input parameters. The model may be trained on data collected from patients, and may receive the body composition parameters described above, as well as other patient and treatment parameters such as one or more of the patient age at the time of diagnosis, sex, smoking status, diabetes status, height, weight, BSA, body max index (BMI), tumour site, cancer staging, genetic risk factors and absolute dose for each drug to be administered. The prediction model may be trained to identify the optimal parameter set for predicting the DLT, and to then use that parameter set to make a prediction. In some embodiments, the prediction model may be trained to output a toxicity rating for an input dosage, which may be in the form of a binary toxicity/no toxicity output. In some embodiments, the prediction model may be trained to output an optimum dosage based on patient parameters. In some embodiments, the prediction model may be trained to output a toxicity score or rating, which may correspond to the likelihood or severity of a predicted toxicity or side effect. In some embodiments, the prediction model may be trained to output a predicted side effect. In some embodiments, the prediction model may be trained to output a predicted severity of a side effect. In some embodiments, the prediction model may be trained to output a predicted duration of a side effect. In some embodiments, the prediction model may be trained to output a score or rating corresponding to a predicted quality of life of the patient should the input drug dosage be administered to the patient. In some embodiments, the prediction model may be configured to output data corresponding to the parameters that were most significant in forming the prediction, such as the specific body composition parameters that most significantly affected the prediction, for example.

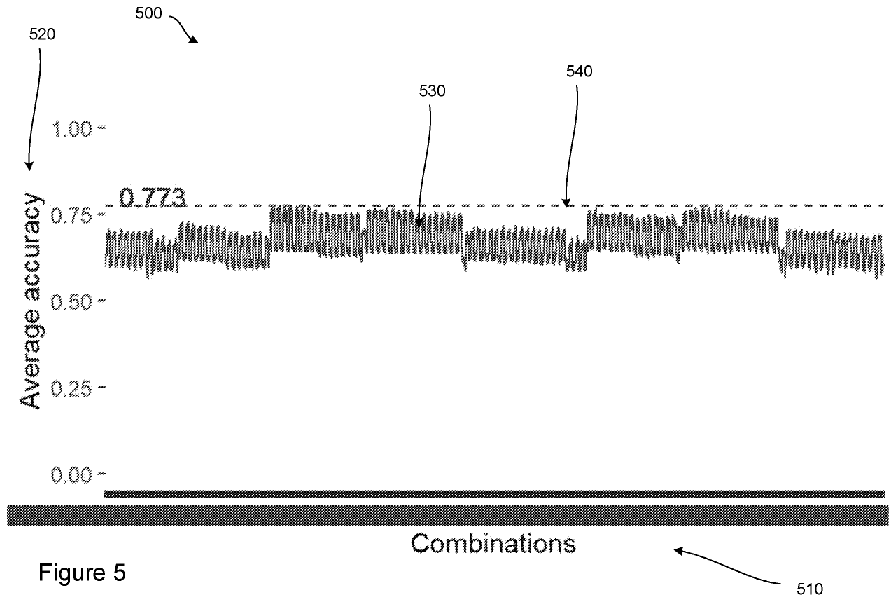

FIG. 5 shows a graph 500 illustrating the accuracy of predictions generated by a prediction model for different combinations of input parameters. The x-axis 510 corresponds to different combinations of input parameters tested, while the y-axis 520 shows the average accuracy of the prediction when using each combination. Line 530 shows the results of the experiment, while line 540 shows the maximum accuracy achieved, being 0.773. For the model being tested, this maximum accuracy was achieved with a set of 10 parameters. The optimal identified parameter set consisted of: LBM, Oxaliplatin, absolute dosage of oxaliplatin, absolute dosage of 5FU, L3 muscle volume, staging_T, height, allmg, L3 SAT radiodensity, age at the time of diagnosis. The following tables provide further accuracy values measured for the experiment:

| | combination | accuracy | sensitivity | specificity |
|---|---|---|---|---|
| 20 | BSA, muscle_volume | 0.676470588 | 0.898181818 | 0.342857143 |
| 23 | BSA, muscle_3d_hu | 0.596405229 | 0.990909091 | 0 |
| 32 | BSA, vat_volume | 0.619281046 | 0.962727273 | 0.1 |
| 34 | BSA, vat_3d_hu | 0.601960784 | 0.990909091 | 0.014285714 |
| 42 | BSA, sat_volume | 0.602287582 | 0.990909091 | 0.014285714 |
| 49 | BSA, sat_3d_hu | 0.602287582 | 0.963636364 | 0.057142857 |
| 57 | LBM, Oxaliplatin | 0.654248366 | 0.927272727 | 0.242857143 |
| 78 | LBM, muscle_volume | 0.715359477 | 0.906363636 | 0.428571429 |
| 81 | LBM, muscle_3d_hu | 0.613398693 | 0.962727273 | 0.085714286 |
| 82 | LBM, vat_volume | 0.608169935 | 0.944545455 | 0.1 |
| 83 | LBM, vat_3d_hu | 0.618954248 | 0.971818182 | 0.085714286 |
| 84 | LBM, sat_volume | 0.618954248 | 0.971818182 | 0.085714286 |
| 85 | LBM, sat_3d_hu | 0.613398693 | 0.953636364 | 0.1 |
| 722 | LBM, muscle_volume, muscle_3d_hu | 0.709803922 | 0.906363636 | 0.414285714 |
| 725 | LBM, muscle_volume, vat_volume | 0.709803922 | 0.906363636 | 0.414285714 |
| 728 | LBM, muscle_volume, vat_3d_hu | 0.698366013 | 0.906363636 | 0.385714286 |
| 732 | LBM, muscle_volume, sat_volume | 0.709803922 | 0.906363636 | 0.414285714 |
| 733 | LBM, muscle_volume, sat_3d_hu | 0.709803922 | 0.906363636 | 0.414285714 |
| 737 | LBM, muscle_3d_hu, vat_volume | 0.608169935 | 0.944545455 | 0.1 |
| 738 | LBM, muscle_3d_hu, vat_3d_hu | 0.613398693 | 0.962727273 | 0.085714286 |
| 739 | LBM, muscle_3d_hu, sat_volume | 0.613398693 | 0.962727273 | 0.085714286 |
| 740 | LBM, muscle_3d_hu, sat_3d_hu | 0.613398693 | 0.962727273 | 0.085714286 |
| 741 | LBM, vat_volume, vat_3d_hu | 0.608169935 | 0.944545455 | 0.1 |
| 742 | LBM, vat_volume, sat_volume | 0.608169935 | 0.944545455 | 0.1 |
| 743 | LBM, vat_volume, sat_3d_hu | 0.61372549 | 0.935454545 | 0.128571429 |
| 744 | LBM, vat_3d_hu, sat_volume | 0.618954248 | 0.971818182 | 0.085714286 |
| 745 | LBM, vat_3d_hu, sat_3d_hu | 0.613398693 | 0.962727273 | 0.085714286 |
| 746 | LBM, sat_volume, sat_3d_hu | 0.630718954 | 0.944545455 | 0.157142857 |

The following table provides performance measures of one specific embodiment that were derived through experimentation:

| Performance | AI | |
|---|---|---|
| measures | model | Interpretation |
| Accuracy | 0.80 | the AI model can correctly classify 80% of |
| | | toxicity and no-toxicity subjects |
| Sensitivity | 0.93 | the AI model can 93% of the times |
| | | correctly identify toxicity subjects |
| Specificity | 0.61 | the AI model has a 61% chance of |
| | | correctly identifying no-toxicity subjects |
| Precision | 0.79 | 79% of toxicity identifications provided by |
| | | the model were accurate |

The prediction tool of some described embodiments utilises one or more of patient demographics, disease, treatment, dosage, toxicity outcomes and CT images (3D) to predict treatment side effects. For example, for a patient suffering from colorectal cancer, the prediction tool may utilise patient demographics, patient colorectal cancer details, chemotherapy treatment, chemotherapy dosage, chemotherapy toxicity outcomes as well as radiomic details from CT images (3D) to predict chemotherapy related toxicities. The CT image data may be received in the Digital Imaging and Communications in Medicine (DICOM) standard, in some embodiments.

In an example, data on colorectal cancer patients treated over the last decade have been used to validate the AI software and have been used to develop the predictive tool as described. Described methods may be able to accurately predict 80% of toxicity and no toxicity patients, in some embodiments.

In the example described above, patient CT scans were accessed, and CT images of patients were downloaded, and relevant patient data was separately identified. The results of patients treated for colon cancer over a 10 year period (2012-2021) were analysed. Of 203 patients with colon cancer treated with chemotherapy such as Oxaliplatin, 120 developed dose limiting toxicities. Of these 203 patients, 80% were accurately predicted by the AI algorithm of some embodiments disclosed herein as likely to develop these complications.

In some embodiments, patient CT scan images may be accessed and processed according to the segmentation model of some described embodiments, and a 3D body composition report may be generated. The prediction model of some described embodiments may utilise patient data to generate a prediction to identify those patients who are at risk of developing toxicities. Described embodiments may be used in clinical research studies to predict toxicity in novel drugs. Described embodiments may be embedded into a health IT cloud where CT images are automatically available to the segmentation model. Clinical patient data may be automatically available to the prediction model, such as by being sent from the EMR system to the cloud software where a prediction report can be generated.

Described embodiments may determine the distribution and quality of various body tissue including muscle, fat, bone and internal body organs in a 3D segment of the body. Every patient has a different distribution of fat, which may be located around one or more specific organs. Some patients may have more fat marbling in a certain area of the body. Quality of tissue such as muscle, fat, bone or organ tissue may be indicated by the brightness/darkness of the areas of muscle, fat, bone and organs as captured in a CT scan (which may be measured in Hounsfield Units) and this may vary in different segments of muscle, fat, bone or organ. Described embodiments may calculate a pattern of fat and muscle from a CT scan. According to some embodiments, the patterns of fat and muscle may be given a score, which may be used as an input parameter to the prediction model in some embodiments. In some embodiments, the patterns of muscle and fat may be allocated to a group or category, which may be used as an input parameter to the prediction model in some embodiments.

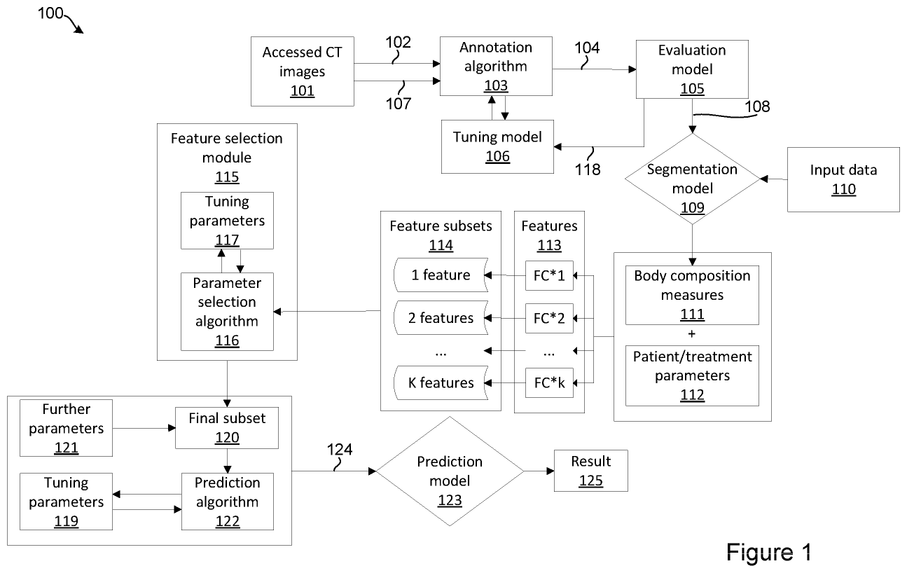

FIG. 1 shows a flowchart of an embodiment of a method 100 for providing a drug dosage prediction according to some embodiments. In the illustrated embodiment, the method commences at 101 with one or more CT images being accessed. Accessing the CT images may include downloading them, retrieving them from a memory storage location, or receiving them from an external computing device, for example. In some embodiments, the accessed CT images may be in the DICOM format.

During a training stage, the images received at 101 constitute a training dataset 102 and are manually annotated into regions representing different tissue types, such as regions representing muscle tissue and regions representing fat. Further annotations may indicate regions of organs and bones.

At 103, an annotation algorithm produces an annotated copy of each CT image received at 101, comprising an annotation of image regions as fat, muscle, bone, organs or other tissue. The annotation algorithm may be an AI algorithm in some embodiments. The generated results 105 are sent to an evaluation model.

At 105, an evaluation module evaluates the accuracy of the output 104. This may be by evaluating how closely the output annotation produced by annotation algorithm 103 resembles a manual annotation of the training images. The manually annotated images may be accessed by evaluation model 105 from a training data set to allow the accuracy of the annotation algorithm 103 to be evaluated. The accuracy estimate 118 is passed back to a tuning module 106, which tunes the internal parameters of the annotation algorithm 103 to improve the accuracy of the results 104, such as by backpropagation and gradient descent. By iteratively passing training data 102 in the form of CT images 101 to annotation algorithm 103 and evaluating its performance, the annotation algorithm 103 can be trained to accurately label CT images.

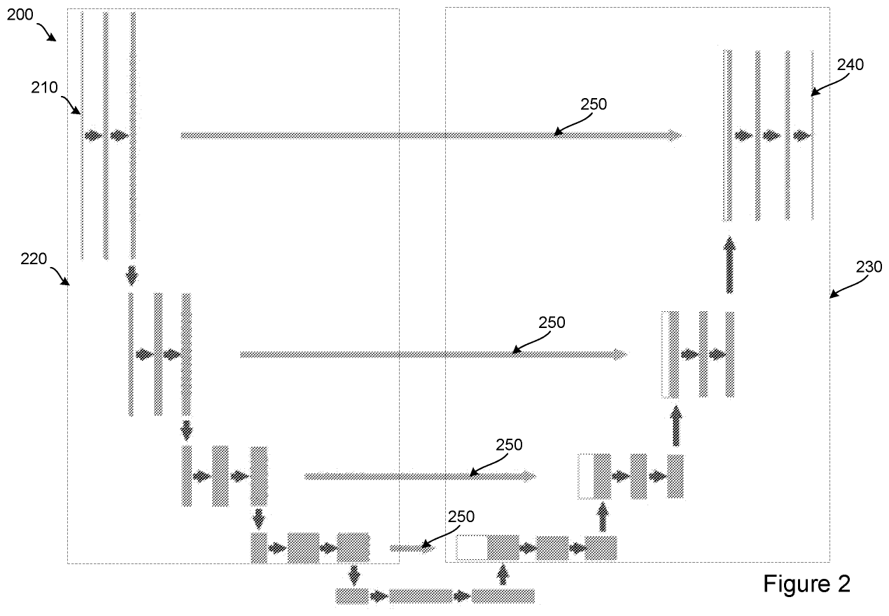

In some embodiments, annotation algorithm 103 comprises a U-net architecture consisting of a contracting path and an expansive path, which gives it a u-shape. The contracting path is a convolutional network that consists of repeated application of convolutions, each followed by a rectified linear unit (ReLU) and a max pooling operation. During the contraction, the spatial information is reduced while feature information is increased. The expansive pathway combines the feature and spatial information through a sequence of up-convolutions and concatenations with high-resolution features from the contracting path. FIG. 2, described in further detail below, illustrates an example architecture 200, noting that other neural networks, convolutional neural networks and types of AI algorithms may equally be used

For generating training dataset 102, a set of CT training scans may first be manually “segmented” or labelled by trained clinicians. These labelled images may be stored and used to train the annotation algorithm 103 as described above. A separate group of CT images may be used to test the annotation algorithm 103. The manual labelling can be performed using software to “colour” in the different body composition types on the CT image so that the AI algorithm is able to learn to self “colour” or segment the images, in some embodiments.

In some embodiments, Hounsfield units may be used in the training process. Hounsfield units are the radiological measurement of how “bright” or how “dark” a pixel is on the CT image. The annotation algorithm 103 may be trained to recognise pattern and body composition based on the position of regions of specific tissues on the scan in combination with how bright or dark the tissue appears as indicated by the Hounsfield units.

The details of an example U-net architecture which may be used by annotation algorithm 103 are described in: Olaf Ronneberger, Philipp Fischer, Thomas Brox, U-Net: Convolutional Networks for Biomedical Image Segmentation, Medical Image Computing and Computer-Assisted Intervention (MICCAI), Springer, LNCS, Vol. 9351:234-241, 2015, available at arXiv: 1505.04597, which is incorporated herein by reference. According to that paper, the input images and their corresponding segmentation maps are used to train the network with the stochastic gradient descent implementation of Caffe. Due to the unpadded convolutions, the output image is smaller than the input by a constant border width. To minimize the overhead and make maximum use of the GPU memory, annotation algorithm 103 may be configured to favor large input tiles over a large batch size and hence reduce a batch of input images to a single tiled image comprising each of the batch of input images. Accordingly, annotation algorithm 103 may be configures to use a high momentum (such as 0.99, for example) such that a large number of the previously seen training samples determine the update in the current optimization step.

The energy function may be computed by a pixel-wise soft-max over the final feature map combined with the cross entropy loss function. The soft-max is defined as

$${p}_{k} ( x ) = exp ( {a}_{k} ( x ) ) / ( {\sum }_{{k}^{′} = 1}^{K} exp ( {a}_{{k}^{′}} ( x ) ) )$$

where aₖ(x) denotes the activation in feature channel k at the pixel position x∈Ω with Ω⊂Z². K is the number of classes and pₖ(x) is the approximated maximum-function. I.e. pₖ(x)≈1 for the k that has the maximum activation aₖ(x) and pₖ(x)≈0 for all other k. The cross entropy then penalizes at each position the deviation of [symbol] (x) from 1 using

$$E = {\sum }_{x ∈ Ω} w ( x ) log ( {p}_{ℓ ( x )} ( x ) ) \qquad ( 1 )$$

where [symbol] : Ω→{1, . . . , K} is the true label of each pixel and w: Ω→R is a weight map that we introduced to give some pixels more importance in the training.

Annotation algorithm 103 may be configured to pre-compute a weight map for each ground truth segmentation to compensate for the different frequency of pixels that may appear from a certain class in the training data set, and to force the network to learn the small separation borders that are introduced between touching cells.

The separation border may be computed using morphological operations. The weight map is then computed as

$$w ( x ) = {w}_{c} ( x ) + {w}_{0} \cdot exp ( - \frac{{( {d}_{1} ( x ) + {d}_{2} ( x ) )}^{2}}{2 {σ}^{2}} ) \qquad ( 2 )$$

where w_{c}: Ω→R is the weight map to balance the class frequencies, d₁: Ω→R denotes the distance to the border of the nearest cell and d₂: Ω→R the distance to the border of the second nearest cell. In our experiments we set w₀=10 and σ≈5 pixels.

In deep networks with many convolutional layers and different paths through the network, a good initialization of the weights is useful. Otherwise, parts of the network might give excessive activations, while other parts never contribute. Ideally the initial weights should be adapted such that each feature map in the network has approximately unit variance. For a network with U-net architecture (alternating convolution and ReLU layers) this can be achieved by drawing the initial weights from a Gaussian distribution with a standard deviation of \sqrt{2/N}, where N denotes the number of incoming nodes of one neuron. E.g. for a 3×3 convolution and 64 feature channels in the previous layer N=9.64-576.

According to some embodiments, the method may tune the hyper-parameters of learning rate, batch size, and number of epochs. Similar or different architectures can be chosen, such as architectures that are available for Google's TensorFlow library.

It is noted that the annotation algorithm 103 may classify areas in a 2-dimensional slice of the CT scan as one or more of muscle, fat, bone or organ. In some examples, the CT slice may be taken from the third lumbar vertebra (L3). However, it may be difficult to determine clinically, whether this particular slice is clinically more relevant that other slices. Therefore, in some embodiments, the method builds a 3-dimensional body model that contains a volume representation of one or more of muscle, fat, bone, organ or other tissue. In one example, this involves processing all slices that contain the L3 vertebra, processing slices from multiple vertebrae, or processing slices from all vertebrae. As a result, the difference in clinical relevance between different CT slices is less relevant and the final output is more accurate.

Once the annotation algorithm 103 is trained (i.e., the parameters are tuned to reduce the difference between the results 104 and the manually labelled images to below a predetermined threshold), the annotation algorithm 103 can be applied to a testing dataset 107, which includes further CT slices that are manually labelled. According to some embodiments, annotation algorithm 103 is not further tuned at this stage, but only applied once to each slice of the testing dataset 107 to generate a result 104 for evaluation by evaluation model 105. If the evaluation model 105 determines that the annotation algorithm 103 has been sufficiently trained, such as by determining that the results of processing the testing database 107 are within a predetermined level of accuracy, the parameters of the trained annotation algorithm 103 may be output as the best fit model 108. The best fit model 108 may be used as the final segmentation model 109. In some embodiments, the segmentation model 109 may continue to be trained and updated with further CT images, which may be CT images from a different area of the body such as a different vertebrae. As described above, the segmentation model 109 may become three-dimensional if more CT slices from the same or different vertebrae are used to bring “height” into the model. In some embodiments, this additional training is performed by annotation algorithm 103 before the best fit model 108 is output to segmentation model 109.

With the segmentation model 109 available, the method 100 calculates body composition measures 111 based on input data 110. The body composition measures 111 are measures that indicate the composition of the body of the patient of whom the images are captured. These measures 111 are quantitivate measures based on the segmentation model 109. The spatial information determined by the segmentation model 109 (which may include labelled areas or volumes), is mapped to one or more numerical measures. These may include one or more of an amount of muscle, an amount of VAT, an amount of SAT, an amount of IMAT, an amount of bone, an amount of organ, a radio-density of muscle, a radio-density of VAT, a radio-density of SAT, a radio-density of bone, and/or a radio-density of organ, for example. The amounts of muscle, VAT, SAT, IMAT, bone or organ may be calculated as respective surface areas in a 2D model (i.e. surface area of muscle, surface area of VAT, surface area of SAT, surface area of IMAT, surface area of bone, surface area of organ) or as respective body volumes in a 3D model (i.e. volume of muscle, volume of VAT, volume of SAT, volume of IMAT, volume of bone, volume of organ).

For the training phase, the body composition measures 111 generated based on the training dataset 102 and testing dataset 107 (and potentially further data from further patients) serve as input parameters to a second classification algorithm 116, as described below. A further input comprises patient and/or treatment parameters 112, which are parameters that are collected during the course of the treatment. These may include further measurements, such as body surface area and lean body mass, data from additional tests such as blood tests, and/or treatment options or parameters, such as absolute dose or type of drug.

At this stage, method 100 may comprise an optional step of feature selection in order to select the best combination of available input features, selected from the available body composition measures 111 and patient/treatment parameters 112. For this purpose, method 100 generates multiple feature combinations 113 with corresponding feature subsets 114. In one example, as the first step of feature selection, 13 parameters including 6 3D body composition measures and 7 other patients and clinical data may be included for selecting the best combination as features 113. In other words, k=13 in this example. Thus, the number of combination of 1 feature=13; the number of combinations of 2 features=78; the number of combinations of 3 features=286; the number of combinations of 4 features=715; . . . the number of combinations of 13 features=1, such that a total of 8191 different combinations can be considered in this example.

These subsets 114 are then provided to a feature selection module 115, comprising a parameter selection algorithm 116 with tuning parameters 117, which creates the final subset 120 of parameters determined by the best fitting model. Feature selection module 115 may use 10-fold cross-validation to train parameter selection algorithm 116 via an iterative model fitting process.

This final subset 120 can be expanded by adding further patient parameters 121 that are not measured but captured by the patient data, such as age, gender, tumour site, etc. In some embodiments, these may be added to the final subset 120 one by one, to test whether their addition improves the accuracy of the final results obtained.

Using the expanded parameter set, a prediction algorithm 122 is tuned using tuning parameters. Prediction algorithm 122 is trained on data from the training dataset 102 and testing dataset 107 together with data from further patients. According to some embodiments, around 1,000 patients may be used to generate a trained prediction algorithm 122.

Once it is determined that prediction algorithm 122 has been sufficiently trained, such as by determining that the results of processing the testing database 107 are within a predetermined level of accuracy, the parameters of the trained prediction algorithm 122 may be output as the best fit model 124. The best fit model 124 may be used as the final prediction model 123. Once both segmentation model 109 and prediction model 123 are trained, these models can be used to generate a result 125 based on input data 110. In some embodiments, the result 125 may correspond to a predicted toxicity of a drug dosage to a patient. In some embodiments, the result 125 may correspond to a predicted safe dosage of a drug to administer to a patient. In some embodiments, the result 125 may correspond to an optimum dosage of a drug to administer to a patient. In some embodiments, the result 125 may correspond to a toxicity rating for an input drug dosage, which may be in the form of a binary toxicity/no toxicity output. In some embodiments, the result 125 may correspond to a toxicity score or rating, which may correspond to the likelihood or severity of a predicted toxicity or side effect. In some embodiments, the result 125 may correspond to a predicted side effect. In some embodiments, the result 125 may correspond to a predicted severity of a side effect. In some embodiments, the result 125 may correspond to a predicted duration of a side effect. In some embodiments, the result 125 may correspond to a score or rating corresponding to a predicted quality of life of the patient should the input drug dosage be administered to the patient. In some embodiments, the result 125 may comprise the parameters that were most significant in forming the prediction, such as the specific body composition parameters that most significantly affected the prediction, for example.

When method 100 is being used to generate a result 125 based in input data 110, the input data is first fed into segmentation model 109 to derive body composition measures 111. Patient/treatment parameters may also be retrieved or derived from input data 110. The final subset 120 of features 113 (as determined during the training process) are fed into prediction model 123 to generate result 125.

In some embodiments, there may be around 30 parameters available for analysis, if not more. It may be too computationally expensive to examine each parameter combination. As an example, there are 33554431 unique combinations of 25 parameters. Method 100 may run different combination tasks in parallel, however computational cost may still be a limitation. As mentioned previously, there are many patient factors which are input into the different models. Using repeated analysis, it is possible to determine which “mixture” of patient factors and to how much/degree they should be incorporated into the “best” algorithm, and these parameters may be stored as final subset 120 to be used in all further processing of input data 110 after model training has been completed. It is possible to add another parameter at 121 and determine whether this improves the algorithm/prediction on an ad-hoc basis.

Different machine learning algorithms were compared, which use all available input parameters to build prediction models to differentiate DLT and no DLT groups in order to determine the optimal machine learning algorithm. The results showed that regularised generalised methods (i.e. Lasso and elastic-net regularized generalized linear models) had the highest performance across different performance indicators, though other methods may be more effective in some situations. The package ‘caret’ enables automated model tuning throughout the training process.

As a second step in the feature selection process, various feature combinations (but not all) were considered. Method 100 may incrementally add the remaining parameters (number=k) to the model and save the model with an improved performance. Method 100 then adds the remaining parameters (number=k−1) . . . until model performance was no longer improved.

FIG. 2 shows an example architecture U-net architecture 200 that may be used to train the annotation algorithm 103 and/or segmentation model 109. Architecture 200 is an example of U-net architecture only, and alternative architectures may be used in some embodiments. Architecture 200 shows an input image 210 being encoded through a series of convolution and pooling layers 220. With each layer, the image being processed gets smaller (indicated by the height of each block), but a number of channels being processed increased (indicated by the thickness of each block). The image is then decoded via a series of up-sampling and convolution layers 230 to arrive at an output segmentation map 240. During up-sampling, the images are concatenated with corresponding images from the pooling steps, shown by skip connections 250.

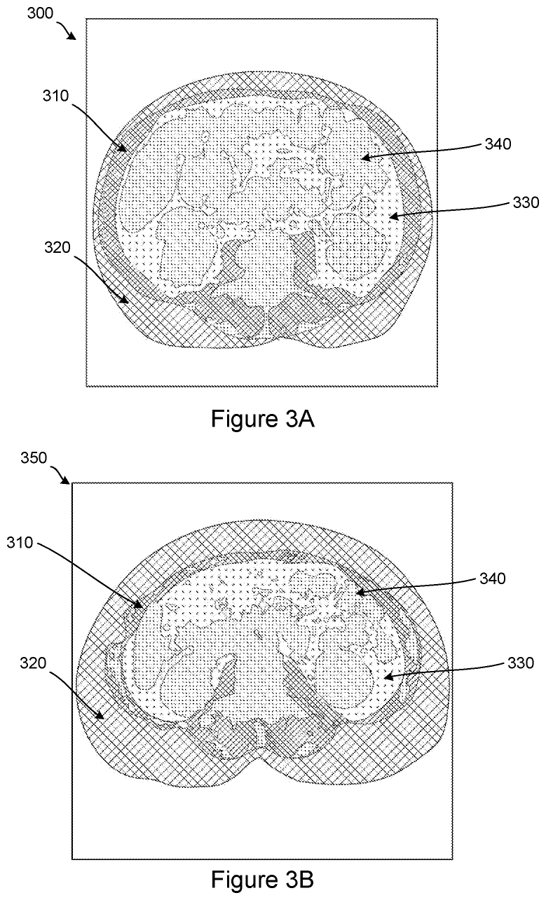

FIG. 3A illustrates an example CT image 300 taken of a first patient, which may be generated by segmentation module 109. FIG. 3A is shaded to indicate various types of identified tissue. Tissue 310 corresponds to muscle; tissue 320 corresponds to sub-cutaneous fat; tissue 330 corresponds to visceral fat, and area 340 corresponds to other tissue such as bone and organs.

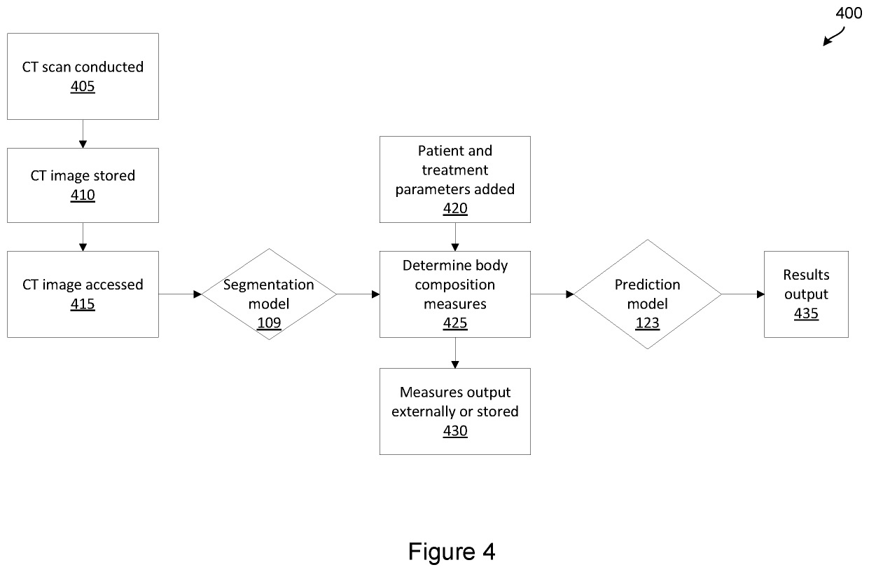

FIG. 3B illustrates a further example CT image 350 taken of a second patient, which may be generated by segmentation module 109. Image 350 has been labelled with the same regions as image 300. However, as visible from the illustration, the second patient has smaller regions of muscle tissue 310, and larger regions of sub-cutaneous fat 320 and visceral fat 330. FIG. 4 illustrates a method 400 for processing patient data according to some embodiments.

At 405, a CT scan of the patient is conducted and at least one CT image is captured.

At 410, the at least one CT image is stored to a memory location, which may be a local storage location on a local computer, or on an external computing device or cloud in some embodiments.

At 415, the at least one CT image is accessed. This may be by downloading the at least one CT image from a memory location, or retrieving it from an external device, for example.

At 109, the trained segmentation model 109 as described above with reference to FIG. 1 is applied to the at least one CT image to generate a 2D or 3D segmented image, where tissue is labelled by the model based on its training. Based on this, body composition parameters are determined at 425. Optionally, these are output or stored at 430, to allow clinician access to these measures.

Patient and/or treatment parameters are accessed and added as input data at 420, and these are passed to prediction model 123 for processing along with the determined body composition measures.

Finally, the trained prediction model 123 is applied to the body composition measures and patient and/or treatment parameters. Prediction model 123 may use the parameters that were selected in the parameter selection steps as described above with reference to FIG. 1. In some embodiments, the segmentation model 109 comprises a convolutional neural network, as such networks are well suited for image classification. Prediction model 123 may use an alternative network as the input is not an image. Therefore, other classification models may perform better.

At 435, results as generated by prediction model 123 are output. The output of the prediction model may be a classification of the input drug dosage with respect to the effect of such a dose on the patient. For example, in some embodiments the output may be one of red, yellow, or green, where
RED—means do not proceed. Chemo toxicity is highly likely to occur. Please consider a lower dose
Yellow—means proceed with caution. Toxicity may be mild but patient should tolerate. Please keep careful review of patient
Green—means it is safe to proceed with the selected dose.

In some embodiments, method 400 may calculate an accurate prescription of a suitable dose, which may be a dose that has the highest effectiveness while reducing the severity, chance or duration of side effects. In other words, the output at 435 may be an accurate chemotherapy dosing.

In some embodiments, prediction model 123 is implemented by a package called ‘caret’, which enables automated model tuning throughout the training process. Caret is the short for Classification And REgression Training and is available in the R statistical toolkit available from the CRAN website https://cran.r-project.org/.

According to some embodiments, the model parameters selected to be used in prediction model 123 may be those with the highest accuracy. The accuracy of the prediction model may be determined by comparing AI classification of two groups (toxicity and no toxicity) with clinical judgement. The indicator for selecting the best model may be accuracy.

Other parameters that may be included into the model, such as at 420, may include cancer characteristics, how aggressive the cancer is (i.e. STAGE), patient demographics (e.g., age, weight, ethnicity), and/or patient blood parameters. Demographic, biometric and cancer characteristic parameters may be obtained from ACCORD. One or more of age at the time of diagnosis, sex, smoking status, and diabetes status may be included as demographic parameters. Biometric parameters may include one or more of height (in cm), weight (in kg), body surface area (BSA) and body mass index (BMI). Cancer characteristic parameters may include one or more of tumour site, cancer staging based on Australian Clinico-pathological Staging (ACPS) System (i.e. staging_acps), and the TNM staging system. Treatment parameters may include example presentations of various regimens (X represents the amount of dose), such as FOLFOX regimen: FOLFOX (5FU: X1 mg over 46 hr, X2 over 3-5 mins/oxaliplatin (Oxa)×3 mg/FA X4 mg).

The model may also incorporate information about genomics—in particular gene mutations which determine ability of the body to metabolise chemotherapy agents. The gene in question may be the dihydropyrimidine dehydrogenase (DPD) enzyme deficiency which is involved with 5-Fluorouracil (5-FU) chemotherapy metabolism.

According to some embodiments, blood test data may be included. This may include data relating to the presence of the dihydropyrimidine dehydrogenase (DPD) enzyme in a patient blood sample.

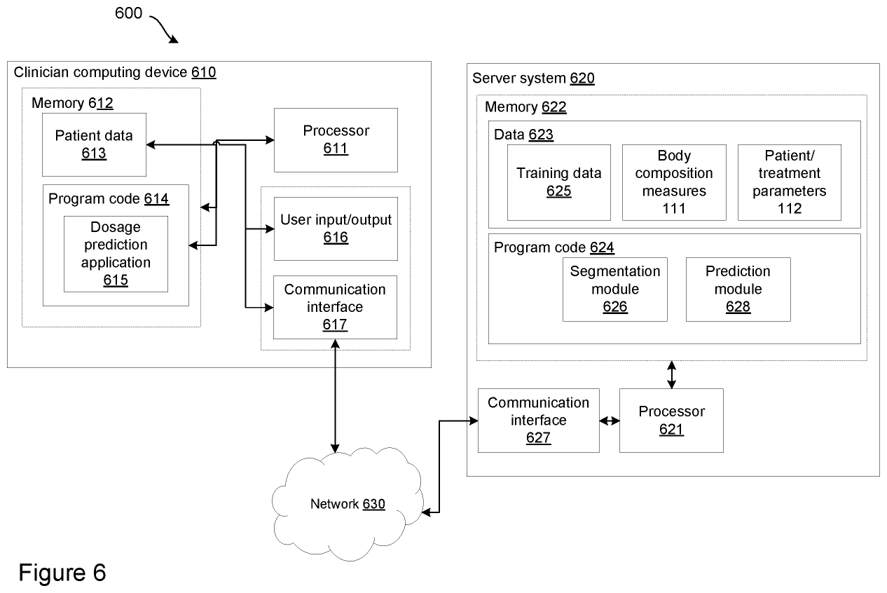

FIG. 6 shows an example system 600 for performing the methods of FIGS. 1 and 4. System 600 includes an arrangement of system components, including hardware and software of systems that may be used to perform the presently disclosed methods. It would be readily understood by the person skilled in the art that the system of FIG. 6 is simply one embodiment of a number of potential embodiments that would be suitable for performing the present methods.

System 600 comprises a clinician computing device 610 which may be controlled by a clinician wishing to generate a drug dosage prediction for a patient. In the illustrated embodiment, system 600 further comprises a server system 620. User computing device 610 may be in communication with server system 620 via a network 630. However, in some embodiments, user computing device 610 may be configured to perform the described methods independently, without access to a network 630 or server system 620.

Clinician computing device 610 may be a computing device such as a personal computer, laptop computer, desktop computer, tablet, or smart phone, for example. Clinician computing device 610 comprises a processor 611 configured to read and execute program code. Processor 611 may include one or more data processors for executing instructions, and may include one or more of a microprocessor, microcontroller-based platform, a suitable integrated circuit, and one or more application-specific integrated circuits (ASICs).

Clinician computing device 610 further comprises at least one memory 612. Memory 612 may include one or more memory storage locations which may include volatile and non-volatile memory, and may be in the form of ROM, RAM, flash or other memory types. Memory 612 may also comprise system memory, such as a BIOS.

Memory 612 is arranged to be accessible to processor 611, and to store data that can be read and written to by processor 611. Memory 612 may also contain program code 614 that is executable by processor 611, to cause processor 611 to perform various functions. For example, program code 614 may include a drug dosage prediction application 615. Processor 621 executing drug dosage prediction application 615 may be caused to perform aspects drug dose prediction methods, such as some steps described above with reference to FIGS. 1 and 4, for example.

According to some embodiments, drug dosage prediction application 615 may be a web browser application (such as Chrome, Safari, Internet Explorer, Opera, or any other alternative web browser application) which may be configured to access web pages that provide functionality via an appropriate uniform resource locator (URL).

Program code 614 may include additional applications that are not illustrated in FIG. 6, such as an operating system application, which may be a mobile operating system if clinician computing device 610 is a mobile device, a desktop operating system if clinician computing device 610 is a desktop device, or an alternative operating system.

Clinician computing device 610 may further comprise user input and output peripherals 616. These may include one or more of a display screen, touch screen display, mouse, keyboard, speaker, microphone, and camera, for example. User I/O 616 may be used to receive data and instructions from a user, and to communicate information to a user.

Clinician computing device 610 may further comprise a communications interface 617, to facilitate communication between clinician computing device 610 and other remote or external devices. Communications module 617 may allow for wired or wireless communication between clinician computing device 610 and external devices, and may use Wi-Fi, USB, Bluetooth, or other communications protocols. According to some embodiments, communications module 617 may facilitate communication between clinician computing device 610 and server system 620 via a network 630, for example.

Network 630 may comprise one or more local area networks or wide area networks that facilitate communication between elements of system 600. For example, according to some embodiments, network 630 may be the internet. However, network 630 may comprise at least a portion of any one or more networks having one or more nodes that transmit, receive, forward, generate, buffer, store, route, switch, process, or a combination thereof, etc. one or more messages, packets, signals, some combination thereof, or so forth. Network 630 may include, for example, one or more of: a wireless network, a wired network, an internet, an intranet, a public network, a packet-switched network, a circuit-switched network, an ad hoc network, an infrastructure network, a public-switched telephone network (PSTN), a cable network, a cellular network, a satellite network, a fibre-optic network, or some combination thereof.

Server system 620 may comprise one or more computing devices and/or server devices (not shown), such as one or more servers, databases, and/or processing devices in communication over a network, with the computing and/or server devices hosting one or more application programs, libraries, APIs or other software elements. The components of server system 620 may provide server-side functionality to one or more client applications, such as dosage prediction application 615. The server-side functionality may include operations such as user account management, login, and other functions such as data sharing functions. According to some embodiments, server system 620 may comprise a cloud based server system. While a single server system 620 is shown, server system 620 may comprise multiple systems of servers, databases, and/or processing devices. Server system 620 may host one or more components of a platform for performing drug dosage prediction according to some described embodiments.

Server system 620 may comprise at least one processor 621 and a memory 622. Processor 621 may include one or more data processors for executing instructions, and may include one or more of a microprocessor, microcontroller-based platform, a suitable integrated circuit, and one or more application-specific integrated circuits (ASIC's). Memory 622 may include one or more memory storage locations, and may be in the form of ROM, RAM, flash or other memory types.

Memory 622 is arranged to be accessible to processor 621, and to contain data 623 that processor 621 is configured to read and write to. Data 623 may store data such as user account data, CT image data, and data relating to machine learning models trained to perform segmentation and prediction functions.

In the illustrated embodiment, data 623 comprises training data 625, body composition measures 111 and patient/treatment parameters 112. While these are illustrated as residing in memory 622 of server system 620, in some embodiments some or all of this data may alternatively or additionally reside in memory 612 of clinician computing device 610, or in an alternative local or remote memory location.

Training data 625 may include CT images 101 including training dataset 102, and testing dataset 107, as described above with reference to FIG. 1. Body composition measures 111 may be stored to data 623 after being generated by segmentation model 109, as described above with reference to FIG. 1. Patient and/or treatment parameters 112 may be stored to data 623 after being received from clinician computing device 610, in some embodiments.

Memory 622 further comprises program code 624 that is executable by processor 621, to cause processor 621 to execute workflows. For example, program code 624 may comprise a server application executable by processor 621 to cause server system 620 to perform server-side functions. According to some embodiments, such as where dosage prediction application 615 is a web browser, the server application may comprise a web server such as Apache, IIS, NGINX, GWS, or an alternative web server. In some embodiments, the server application may comprise an application server configured specifically to interact with dosage prediction application 615. Server system 620 may be provided with both web server and application server modules.

Program code 624 may also comprise one or more code modules, such as one or more of a segmentation module 626 and a prediction module 628. Segmentation module 626 and prediction module 628 may be configured to cause processor 621 to execute the functions of the segmentation model 109 and the prediction model 123, as described above with reference to FIGS. 1 and 4.

Segmentation module 626 and prediction module 628 may be software modules such as add-ons or plug-ins that operate in conjunction with the dosage prediction application 615 to expand the functionality thereof. In alternative embodiments, modules 626 and/or 628 may be native to dosage prediction application 615. In still further alternative embodiments, modules 626 and/or 628 may be a stand-alone applications (running on user computing device 610, server system 620, or an alternative server system (not shown)) which communicate with the dosage prediction application 615, such as over network 630.

Modules 626, and 628 have been described and illustrated as being part of/installed on the server system 620, and may be configured as an add-on or extension to a server application, a separate, stand-alone server application that communicates with the server application, or a native part of the server application. Inputs, such as user interactions, patient data and/or CT images, may be provided and/or received at/by the clinician computing device 610, and then transferred to server system 620, such that the dosage prediction method may be performed by the components of the server system 620.

In some alternative embodiments (not shown), the functionality provided by one or more of modules 626 and/or 628 could alternatively be provided by clinician computing device 610, based on locally or remotely stored data. One or more of modules 626 and/or 628 may reside as an add-on or extension to dosage prediction application 615, a separate, stand-alone application that communicates with dosage prediction application 615, or a native part of dosage prediction application 615.

In alternate embodiments (not shown), all functions may be performed by the server system 620. Or, in some embodiments, an application programming interface (API) may be used to interface with the server system 620 for performing the presently disclosed techniques.

Server system 620 may also comprise a communications interface 627, to facilitate communication between server system 620 and other remote or external devices. Communications module 627 may allow for wired or wireless communication between server system 620 and external devices, and may use Wi-Fi, USB, Bluetooth, or other communications protocols. According to some embodiments, communications module 627 may facilitate communication between server system 620 and clinician computing device 610, for example.

Server system 620 may include additional functional components to those illustrated and described, such as one or more firewalls (and/or other network security components), load balancers (for managing access to the server application), and or other components.

A retrospective study was performed on stage 3 colon cancer patients receiving oxaliplatin following surgery at a single tertiary institution. Validated AI algorithms were used to calculate body composition from staging CT scans. Oxaliplatin dose/lean body mass cut-points for toxicities were derived from receiver operating characteristics analysis.

Between 2012-2021, 129 patients were identified (male 53%, mean age 58 (25-80 years)). Surgical procedure primarily included right hemi/extended right hemicolectomy (n=55), anterior resection (n=44), Hartmann's procedure (n=12) and others (n=18).

Dose-limiting toxicity was experienced in 84 (65.1%) patients and was higher in females (p=0.025). Females had significantly lower muscle indices and higher adiposity (p<0.001). BSA was similar in both the DLT and non-DLT groups. Lean body mass and BSA had a weak correlation (R2=0.514, p=0.001). The optimal oxaliplatin cut point was identified as 3.41 mg/kg lean body mass. Within the first four cycles of chemotherapy, 37 patients experienced DLT, of which 29 (78%) received a dose equal to or greater than the predicted cut-point (p=0.05).

This demonstrates the use of AI-automated body composition measurements to predict the risk of drug-related toxicities was able to predict early treatment toxicity successfully.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.

1. A method for predicting a toxicity of a drug to be administered to a patient, the method comprising:
accessing at least one computed tomography (CT) torso slice associated with the patient;
using a trained artificial intelligence (AI) segmentation model to perform a labelling process of the at least one CT scan;
determining at least one body composition parameter based on the at least one labelled CT slice;
receiving at least one parameter associated with the patient;
receiving at least one dosage parameter associated with a drug to be administered to the patient; and
using a trained AI prediction model, generating an output based on the at least one body composition parameter, the at least one demographic parameter and the at least one dosage parameter, the output corresponding to a predicted toxicity of the drug to the patient.

2. The method of claim 1, wherein the method further comprises training the AI prediction model.

3. The method of claim 1 or 2, wherein the labelled CT slices include one or more or all of labelled areas of muscle, visceral adipose tissue (VAT), subcutaneous adipose tissue (SAT), intermuscular/intramuscular adipose tissue (IMAT), bone and organ.

4. The method of any one of the preceding claims, wherein the body composition parameter is at least one or more or all of an amount of muscle, an amount of VAT, an amount of SAT, an amount of IMAT, an amount of bone, an amount of organ, a radio-density of muscle, a radio-density of VAT, a radio-density of SAT, a radio density of IMAT, a radio-density of bone or a radio-density of organ.

5. The method of any one of claims 1 to 4, wherein the drug is a chemotherapy drug used to treat one of breast cancer, lung cancer, liver cancer, colon cancer, bowel cancer, prostate cancer, colorectal cancer, and pancreatic cancer.

6. The method of any one of claims 1 to 5, wherein the parameter associated with the patient comprises a parameter associates with a treatment being administered to the patient.

7. The method of any one of claims 1 to 6, wherein the parameter associated with the patient comprises a parameter obtained from a blood test.

8. A method for training an artificial intelligence model to assess a drug dosage for minimising a toxic reaction to the drug, the method comprising:
receiving a sample of labelled CT torso slices associated with a sample of patients;
determining at least one body composition parameter based on each labelled CT slice;
identifying a set of prediction parameters for each patient, the prediction parameters including the at least one body composition parameter, at least one patient demographic parameter and at least one dosage parameter;
for each combination of the prediction parameters, performing a model fitting process to cause the model to generate a predicted toxicity rating for each patient;
for each combination of the prediction parameters, comparing the predicted toxicity rating with a measured toxicity rating to determine the accuracy of the combination of prediction parameters; and
selecting the optimal combination of prediction parameters, wherein the optimal combination of prediction parameters are the combination of prediction parameters that result in the highest accuracy.

9. The method of claim 8, wherein the labelled CT slices include one or more or all of labelled areas of muscle, VAT, SAT, IMAT, bone and organ.

10. The method of claim 8 or 9, wherein the body composition parameter is at least one or more or all of an amount of muscle, an amount of VAT, an amount of SAT, an amount of IMAT, an amount of bone, an amount of organ, a radio-density of muscle, a radio-density of VAT, a radio-density of SAT, a radio-density of IMAT, a radio-density of bone or a radio-density of organ.

11. The method of any one of claims 8 to 10, wherein the method further comprises using the trained AI model to perform labelling of a sample of CT slices to generate the sample of labelled CT slices.

12. The method of any one of the preceding claims, wherein the CT slice is from the abdomen.

13. The method of any one of the preceding claims, wherein between two and one thousand CT slices are received.

14. A method of minimising a toxic reaction to a drug in a patient, the method comprising:
i) determining the amount of muscle, visceral adipose tissue (VAT), subcutaneous adipose tissue (SAT) and intermuscular/intramuscular adipose tissue (IMAT) in at least a part of the torso of the patient,
ii) determining the risk of a toxic reaction in the patient to the drug at least based on i), and
iii) reducing a standard recommended dose of the drug if it is determined that the patient is at risk of a toxic reaction.

15. The method of claim 14 which further comprises administering the drug.

16. The method of claim 14 or claim 15, wherein step i) is performed using the method of any one of claim 1 to 7, 12 or 13.

17. The method of any one of claims 14 to 16, wherein the drug is a chemotherapy drug used to treat one of breast cancer, lung cancer, liver cancer, colon cancer, bowel cancer, prostate cancer, colorectal cancer, and pancreatic cancer.

18. A non-transient computer-readable storage medium storing instructions which, when executed by a processing device, cause the processing device to perform the method of any one of claims 1 to 17.

19. A computer system configured for generating text, the computer system comprising:
a processor; and
the storage medium of claim 18.
